(19) United States
(12) Patent Application Publication
(10) Pub. No.: US 2017/0027990 A1
(43) Pub. Date: Feb. 2, 2017

(54) METHOD OF TREATING MENTAL STATE IN PATIENTS AFTER ISCHEMIC BRAIN INJURY WITH ISCHEMIC TOLERANT ALLOGENEIC MESENCHYMAL BONE MARROW CELLS

(71) Applicant: Nikolai Tankovich, San Diego, CA (US)

(72) Inventor: Nikolai Tankovich, San Diego, CA (US)

(73) Assignee: Stemedica Cell Technologies, Inc., San Diego, CA (US)

(21) Appl. No.: 14/832,887

(22) Filed: Aug. 21, 2015

Related U.S. Application Data
(60) Provisional application No. 62/197,424, filed on Jul. 27, 2015.

(51) Int. Cl. A61K 35/28; A61K 9/00

(52) U.S. Cl. CPC A61K 35/28; A61K 9/0019

(57) ABSTRACT

A method of treating the mental state of subjects with significant neurological, functional and/or motor impairment from an ischemic stroke. The method comprises a step of intravenously dosing ischemic tolerant allogeneic mesenchymal bone marrow cells itMSCs to subjects with ischemic stroke.

This application claims the benefit of Provisional Patent application US62/197,424 filed Jul. 27, 2015.

### FIELD OF INVENTION

The invention is concerned with a method of treating the mental state of patients with neurologic, functional, and/or motor deficits after ischemic stroke by intravenously administering allogeneic bone-marrow derived mesenchymal stem cells (itMSCs).

### BACKGROUND

### Neurodegenerative Diseases

Neurodegenerative diseases are becoming more prevalent as the world's population ages. It has been estimated that by the year 2040, the number of people affected by a neurodegenerative disease will double to approximately 40 million people worldwide. In addition, this group of diseases is generally considered to be progressive and incurable, and currently have no approved treatment options that change the long-term prognosis or are curative. The three most prevalent chronic, age-related neurodegenerative diseases are Stroke, Parkinson's disease, and Alzheimer's disease, and there are currently significant research efforts focused on the prevention and treatment of these devastating neurodegenerative diseases that together create a vast global unmet medical need.

Stroke

Stroke remains a major global healthcare problem. Recent data compiled by the American Heart Association (AHA) for 2008 show that the annual incidence of new or recurrent stroke in the United States is about 780,000, with approximately 600,000 of these strokes being first attacks. Among adults age 20 and older, the estimated prevalence of stroke in 2005 was 5.8 million in the United States, resulting in >150,000 deaths annually, with 4.8 million stroke survivors alive today.1 Stroke ranks as the country's third leading cause of death, behind only cancer and heart disease. Stroke data indicates that the incidence may be increasing. From 1988 to 1997, the age-adjusted stroke hospitalization rate grew 18.6% (from 560 to 664 per 100 000), while total stroke hospitalizations increased 38.6% (from 592,811 to 821,760 annually).

Consistent with the pathophysiology and symptoms of stroke, stroke is a leading cause of adult impairment, with 20% of stroke survivors requiring institutional care after 3 months and up to 30% being severely and permanently disabled. The only approved treatments of acute ischemic stroke involve restoring blood flow to the affected region by using thrombolytics or mechanical devices that physically remove clots. However, the use of thrombolytics (tPA) is limited due to the therapeutic window of <3-6 hours post onset of stroke symptoms such that only a small fraction of stroke patients receive this therapy, thus effective prevention remains the best treatment for reducing the burden of stroke. Following the completion of a stroke, there is little therapy to offer patients to promote recovery other than physical, occupational, and speech therapy.

Stem Cells

Research efforts in the field of stem cell biology have accelerated and intensified during the last few decades and have provided important information on the developmental roles and pathophysiologic responses of stem cells. Stem cells can be found in many tissues (e.g., bone marrow, brain, adipose tissue, muscle, and eye) and can be isolated from many different tissue sources (e.g., embryo, fetus, placenta, umbilical cord blood). The advances in our understanding of stem cell biology have generated significant interest in the therapeutic potential of exogenous stem cell administration.

Allogeneic Mesenchymal Bone Marrow Cells (MSCs)

Mesenchymal stem cells have been isolated from a number of sources, including bone marrow, adipose tissue, peripheral blood, amniotic fluid, fetal liver, and umbilical cord blood. The MSCs manufactured for this study are a subset of non-hematopoietic stem cells derived from the bone marrow of healthy, adult donors, and have the ability to migrate and differentiate into multiple cell types depending on the local environment and extracellular matrix, and include cartilage, bone, adipose tissue, vasculature and neurons. As described below, the MSCs manufactured for this study were cultured and expanded in hypoxic (low-oxygen) conditions, referred to herein as ischemic tolerant mesenchymal stem cells, itMSCs. Several recent reports demonstrate that human MSCs derived from adult human bone marrow can differentiate in culture to neuronal stem cells that have all the neuroectodermal markers appropriate for this lineage, including nestin and otxl.

### BRIEF DESCRIPTION OF FIGURES

FIG. 1 Dosing Schedule 0.5 million cells per kilogram

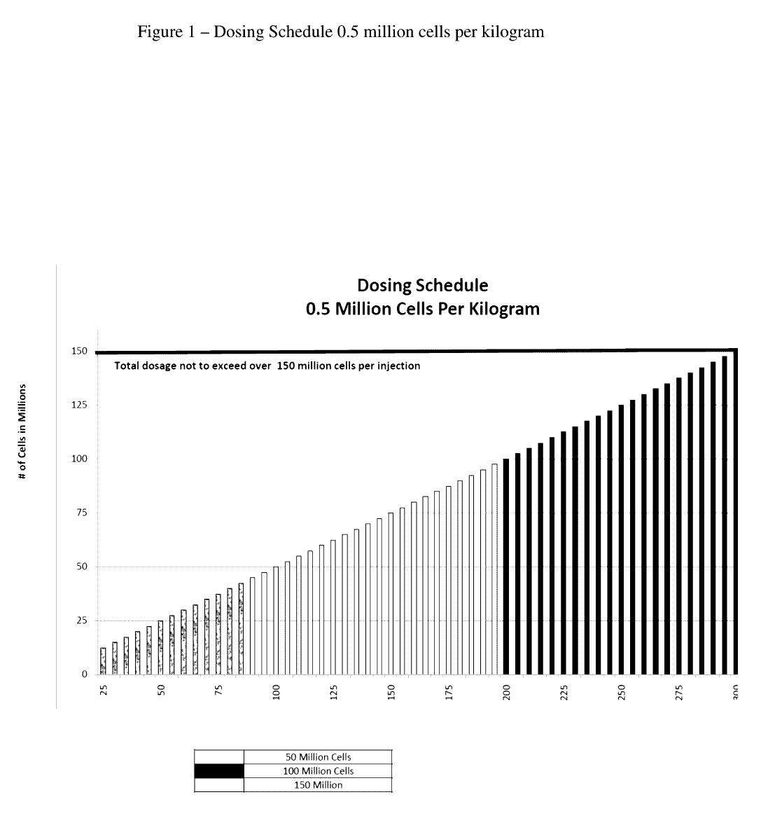

FIG. 2 Dosing Schedule 1.0 million cells per kilogram

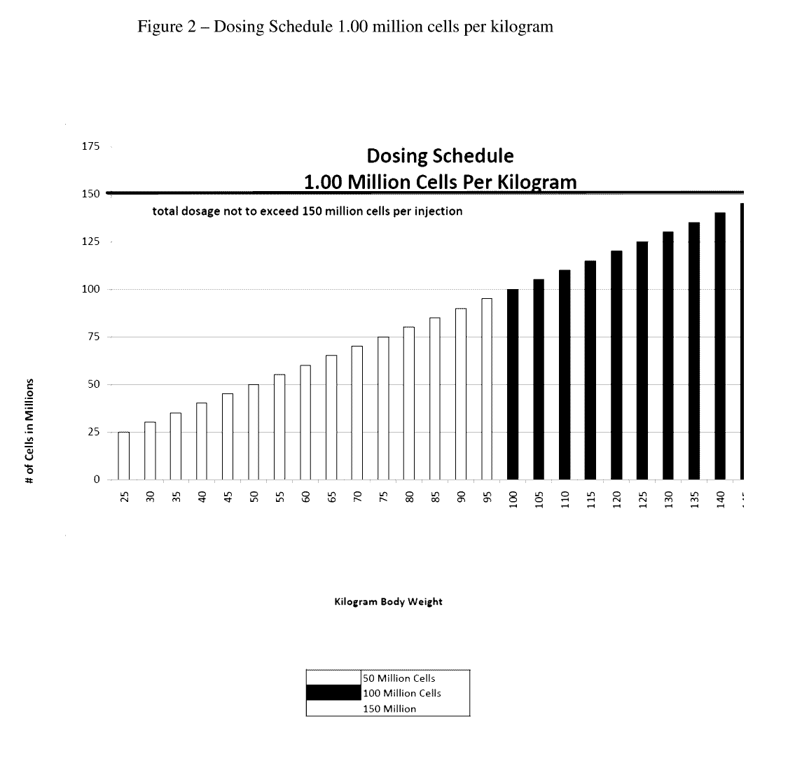

FIG. 3 Dosing Schedule 1.5 million cells per kilogram

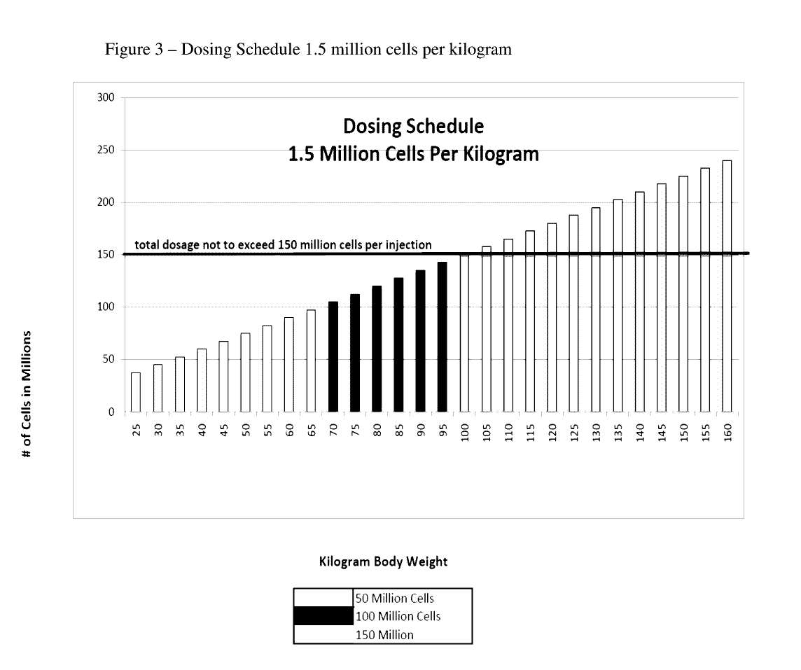

### DETAILED DESCRIPTION

The present invention is directed to a method of treating the mental state of subjects with significant neurological, functional and/or motor impairment from an ischemic stroke. The method comprises a step of intravenously dosing ischemic tolerant allogeneic mesenchymal bone marrow cells itMSCs to subjects with ischemic stroke. In another aspect, the present invention is directed to treatment of the mental state with itMSCs of neurologic, functional, and/or motor deficits in patients with ischemic stroke.

Subject Patients.

Each of the cohorts neurologically assessed or examined for neurologic, functional, and motor deficits in patients contained seventeen or eighteen subjects with ischemic stroke.

A screening evaluation to determine if the subject was a suitable candidate for participation in this study was conducted prior to enrollment and treatment by the method of the invention.

The patients were evaluated upon admission according to the schedule of medical assessments to ensure eligibility of the subjects. Intravenous dosing of the itMSCs was performed on the morning of Day 1. Subjects remained in the hospital until stable and were regularly monitored for safety. Subjects that were clinically stable were discharged into the care of a responsible individual.

Follow-up visits for assessment of safety occurred daily for 3 days (days 2, 3, 4), again in one week (day 10) and at 1, 3, 6, 9, and 12 months post-treatment.

Adverse events, concomitant medications, vital signs, clinical laboratory test results, physical examinations, neurologic examinations, and neurologic assessment tools were collected or conducted at follow-up visits according to the schedule of assessments.

| | Screen | Tx. | Follow-up | | | | | | | | | Early |
|---|---|---|---|---|---|---|---|---|---|---|---|---|
| | Day −30 | Day | Day | Day | Day | Day | Month 1 | Month 3 | Month 6 | Month 9 | Month 12 | Termi- |
| Assessments | to −1 | 1 | 2⁸ | 3 | 4 | 10 | (Day 30) | (Day 90) | (Day 180) | (Day 270) | (Day 360) | nation |
| Written Informed Consent | X | | | | | | | | | | | |
| Inclusion/Exclusion Criteria | X | X | | | | | | | | | | |
| Medical History | X | X | | | | | | | | | | |
| Standard Physical Exam | X | X | | | X | X | X | X | X | X | X | X |
| Height and Weight | X | X¹ | | | | | X¹ | X¹ | X¹ | X¹ | X¹ | X¹ |
| Neurologic Exam | X | X | | | X | X | X | X | X | X | X | X |
| Neurologic Assessment Tools² | X | | | | | | X | X | X | X | X | X |
| Blood Pressure, Pulse, | X | X³ | X | X | X | X | X | X | X | X | X | X |
| and Oral temperature | | | | | | | | | | | | |
| 12-lead Electrocardiogram | X | X | | | | | X | | X | | X | X |
| HIV, HbsAg, HCAb tests | X | | | | | | | | | | | |
| Serum Pregnancy Test | X | | | | | | | | | | | |
| Urine Pregnancy Test (local) | | X | | | | | | | | | | |
| CT with and without contrast | X | | | | | | | | X | | X | X |
| of chest, abdomen and pelvis | | | | | | | | | | | | |
| Brain CT without contrast | X* | | | | | | | | | | | |
| (*unless taken within 2 | | | | | | | | | | | | |
| mo of enrollment) | | | | | | | | | | | | |
| Urine dipstick | X | X | | | | | | | | | X | |
| pT, APTT and LFT's | X | X⁵ | X | X | X | X | X | X | X | X | X | X |
| CBC with differential | X | X⁵ | X | X | X | X | X | X | X | X | X | X |
| Serum Chemistry | X | X⁵ | X | X | X | X | X | X | X | X | X | X |
| Clinical Treatment Day | | X | | | | | | | | | | |
| IgA, IgE, IgG, IgM and | | X⁶ | | | | | | X | X | | X | |
| Lymphocyte Prolifera- | | | | | | | | | | | | |
| tion Panel | | | | | | | | | | | | |
| Bovine serum skin test | | X | | | | | | | | | | |
| Intravenous Mesenchymal | | X | | | | | | | | | | |
| Bone Marrow Cell Infusion | | | | | | | | | | | | |
| Adverse Events⁷ | | X | X | X | X | X | X | X | X | X | X | X |
| Concomitant Medications | X | X | X | X | X | X | X | X | X | X | X | X |

Study Endpoints

Primary

The primary endpoint of safety and tolerability of treatment with itMSCs during the twelve-month study period were determined by the incidence and severity of adverse events, clinically-significant changes on clinical laboratory tests, vital signs, physical and neurologic examinations.

Secondary

The change from baseline on neurologic and physical exam plus the following neurologic assessment tools were conducted at 1, 3, 6, 9, 12 months post-treatment, as available:

National Institutes of Health Stroke Scale (NIHSS)

Mini Mental Status Exam (MMSE)

Barthel Index (BI)

The Geriatric Depression Scale (GDS)

Subject Selection

Subjects were selected using the following inclusion and exclusion criteria.

Inclusion Criteria:
1. Males and females ≧18 years of age
2. Clinical diagnosis of ischemic stroke for longer than 6 months
3. Brain C T scan consistent with patient's functional impairment (unless CT or MRI taken within 2 months of enrollment)
4. Significant functional or neurologic impairment related to the diagnosis in #2 that confines the subject to a wheelchair or requires the subject to have home nursing care or assistance with general activities of daily living
5. No substantial improvement in neurologic or functional deficits for the 2 months prior to enrollment in the study per medical history
6. NIHSS score between 6-20
7. Life expectancy greater than 12 months
8. Ability to understand and provide signed informed consent, or have a designated legal guardian or spouse willing to make such decisions on the subject's behalf
9. Reasonable expectation that patient will receive standard post-treatment care and attend all scheduled safety follow-up visits
10. Adequate organ function as defined by the following criteria:
   Serum aspartate aminotransferase (AST)≦2.5×upper limit of normal (ULN).
   Serum glutamate-oxalate transferase (SGOT)≦2.5×ULN
   Serum alanine aminotransferase (ALT)≦2.5×ULN
   Serum glutamate-pyruvate transferase [SGPT])≦2.5×ULN
   Total serum bilirubin≦1.5×ULN;
   Prothrombin time (PT) and partial thromboplastin time (PTT)≦1.5×ULN;
   Serum albumin≧3.0 g/dL;
   Absolute neutrophil count (ANC)≧1500/μL;
   Platelets≧150,000/μL;
   Hemoglobin≧9.0 g/dL;
   Serum creatinine≦1.5×ULN;
   Serum amylase or lipase≦1.0×ULN.

Exclusion Criteria:
1. History of uncontrolled seizure disorder
2. Presence of immune deficiency disorder
3. History of cancer within the past 5 years
4. Presence of any other clinically-significant medical condition, psychiatric, or laboratory abnormality that in the judgment of the Investigator or Sponsor for which participation in the study would pose a safety risk to the subject
5. Findings on baseline CT suggestive of subarachnoid or intracerebral hemorrhage within past 12 months. Cerebral neoplasm by approval on individual case basis
6. Participation in another study with an investigational drug or device within 3 months prior to treatment
7. History within the past year of drug or alcohol abuse
8. Females known to be pregnant, lactating or having a positive pregnancy test (will be tested during screening) or planning to become pregnant during the study
9. Allergies to Bovine and Porcine products

Study Treatment

All subjects were dosed at a single dose level that was at or below the highest dose determined to be safe in the safety phase of this study.

Administration of itMSCs

On Day 1 an intravenous line was placed into an appropriate vein on the upper extremity with 0.9% sodium chloride running to keep the vein open. Within 1 hour prior to infusion, the appropriate number of itMSCs was diluted into 100 ml of 0.9% sodium chloride and infused intravenously within 50 minutes.

Prior to treatment a skin test to bovine serum was administered using 0.1 ml aliquot of the Stem Cell Media (Bovine Growth Serum Hyclone). If the patient had a reaction during the infusion, the infusion was interrupted or slowed according to the degree of reaction.

The patient remained remained in the hospital for observation for 24 hours. When clinically stable, the patient was discharged home with a responsible adult, and was monitored according to the schedule of assessments (Appendix B).

Concomitant Therapy and Clinical Practice

Patients continued all of their regular medications unless contraindicated. Rehabilitation (i.e., Physical, speech, and/or occupational therapy) were instituted upon returning home, as clinically indicated. Specific rehabilitation interventions and modalities were based upon the subject-specific evaluation findings, impairments, treatment plans, and goals.

Statistical Methods

The efficacy analyses included subjects that received the full dose of itMSCs and had the appropriate follow-up ratings completed per the schedule of assessments (i.e., Efficacy Evaluable Population). Efficacy of the neurological assessment tools: NIHSS, MMSE, BI, and MS were employed across the entire efficacy evaluable population.

The physical and symptomatic responses observed from this study of the actions of the MSCs on the central nervous system supported the present method of treating to obtain the benefits of itMSCs regenerative therapy in neurologic and neurodegenerative disease.

### OBJECTIVES OF STUDY

### Primary Objective

To assess the safety and tolerability of ischemic tolerant allogeneic mesenchymal bone marrow cells (itMSCs) administered intravenously to patients with ischemic stroke.

Secondary Objectives

To assess the clinical effects on the patient's mental state of treatment with itMSCs on neurologic, functional, and motor deficits in patients with ischemic stroke.

Mental State and Neurologic Exam

It should be understood that the term “mental state” as used herein means a patient's phenotype as reflected in the patient's performance on neurologic exams compared to baseline measurement on mental state assessment tools, such as but not restricted to National Institute of Health Stroke Scale (NIHSS), Mini Mental Status Examination (MMSE), The Barthel Index (BI), and Geriatric Depression Scale (GDS).

In clinical practice, mental state examinations are appraisals of the appearance, behavior, mental functioning and overall demeanor of a person. In some ways it reflects a “snapshot” of a person's psychological functioning or a person's capacity to function at a given point in time.

A typical mental state examination considers or appraises the following domains:

Appearance: A person's appearance can provide useful clues into their quality of self-care, lifestyle and daily living skills in terms of distinctive features, clothing, grooming, and hygiene.

Behavior: As well as noting what a person is actually doing during the examination, attention is paid to behaviors typically described as non-verbal communication. These can reveal much about a person's emotional state and attitude. Appraisal is made of the patient's facial expression, body language and gestures, posture, eye contact response to the assessment itself, rapport and social engagement, level of arousal (e.g. calm, agitated), anxious or aggressive behavior, psychomotor activity and movement (e.g. hyperactivity, hypoactivity), unusual features (e.g. tremors, or slowed, repetitive, or involuntary movements)

Mood and affect: Affect refers to immediate expressions of emotion, while mood refers to emotional experience over a more prolonged period of time. Affect is measured in term range (e.g. restricted, blunted, flat, expansive), appropriateness (e.g. appropriate, inappropriate, incongruous), stability (e.g. stable, labile). Mood could appear as happiness (eg, ecstatic, elevated, lowered, depressed), irritability (e.g. explosive, irritable, calm), stability.

Speech: Speech described behaviorally and described by its content is particularly revealing feature of a person's presentation. Unusual speech is sometimes associated with mood and anxiety problems, schizophrenia, and organic pathology. The following are typical measures: speech rate (e.g. rapid, pressured, reduced tempo), volume (e.g. loud, normal, soft), tonality (e.g. monotonous, tremulous), quantity (e.g. minimal, voluble), ease of conversation.

Cognition: This refers to a person's current capacity to process information and is important because it is often sensitive to mental health problems. The following are observed: level of consciousness (e.g. alert, drowsy, intoxicated, stuporose), orientation to reality (often expressed in regard to time/place/person—e.g. awareness of the time/day/date, where they are, ability to provide personal details), memory functioning (including immediate or short-term memory, and memory for recent and remote information or events), literacy and arithmetic skills, visuospatial processing (e.g. copying a diagram, drawing a bicycle), attention and concentration (e.g. observations about level of distractibility, or performance on a mentally effortful task—e.g. counting backwards by 7's from 100), general knowledge, language (e.g. naming objects, following instructions), ability to deal with abstract concepts (e.g. describing conceptual similarity between two things).

Thoughts: A person's thinking is generally evaluated according to their thought content or nature, and thought form or process. Content measures include: delusions (rigidly held false beliefs not consistent with the person's background), overvalued ideas (unreasonable belief, e.g. a person with anorexia believing they are overweight), preoccupations, depressive thoughts, self-harm, suicidal, aggressive or homicidal ideation, obsessions (preoccupying and repetitive thoughts about a feared or catastrophic outcome, often indicated by associated compulsive behavior), anxiety (generalized, i.e. heightened anxiety with no specific referent; or specific, e.g. phobias)

Process: Thought process refers to the formation and coherence of thoughts and is inferred very much through the person's speech and expression of ideas. Observed are: highly irrelevant comments (loose associations or derailment), frequent changes of topic (flight of ideas or tangential thinking), excessive vagueness (circumstantial thinking), nonsense words (or word salad), pressured or halted speech (thought racing or blocking)

Perception: Screening for perceptual disturbance is critical for detecting serious mental health problems like psychosis or severe anxiety, and mood disorders. It is also important in trauma or substance abuse. Perceptual disturbances are typically marked and may be disturbing or frightening. Disturbances include: Dissociative symptoms like derealization (feeling that the world or one's surroundings are not real), and depersonalization (feeling detached from oneself. Illusions include the person perceives things as different to usual, but accepts that they are not real, or that things are perceived differently by others. Hallucinations are probably the most widely known form of perceptual disturbance, hallucinations are indistinguishable by the sufferer from reality, could include command hallucinations (voices telling the person to do something) should be investigated, or associated to degrees of fear and/or distress associated with the hallucinations.

Insight & Judgement: Insight involves acknowledgement of a possible mental health problem, understanding of possible treatment options and ability to comply with these, and ability to identify potentially pathological events (e.g. hallucinations, suicidal impulses) Judgement refers to a person's problem-solving ability in a more general sense, and can be evaluated by exploring recent decision-making or by posing a practical dilemma (e.g. what should you do if you see smoke coming out of a house?)

### STUDY DESIGN

### Subjects of the Study

The subjects of this study had significant functional and/or neurologic impairment from an ischemic stroke. The first part 1 was a dose escalation design. The second part determined efficacy of the present invention on a cohort based on the safety findings of Part 1.

Part 1 of the study consisted of three dose cohorts of subjects (n=5 per dose cohort) with ischemic stroke who were enrolled sequentially in an escalating dose manner, (see Appendix A), with subjects receiving the same weight based dosage of mesenchymal bone marrow cells:
Dose cohort 1: 0.5 million mesenchymal cells per kilogram body weight
Dose cohort 2: 1.0 million mesenchymal cells per kilogram body weight
Dose cohort 3: 1.5 million mesenchymal cells per kilogram body weight

It was determined to be safe to continue the study after the three cohorts were doses and observed.

For determining efficacy, the second part of the study enrolled an additional 18 subjects with ischemic stroke at for dosing at or below the highest safe dose level as determined from Part 1. Adverse events, concomitant medications, and assessments of neurologic status were recorded at each follow-up visit or contact from the patient and/or patient's caretaker, following discharge from the hospital daily on day 2, 3, 4 and 10 post-treatment, as shown in the above Schedule of Assessments. Complete neurological examinations and neurologic assessment tools were used to assess disability and functional status at baseline (prior to treatment), and at the visits scheduled at 1, 3, 6, 9, and 12 months post treatment. Computerized tomography (CT) was conducted at baseline only, unless CT or MRI were collected within 2 months of enrollment.

Upon completion of the cell transplantation, patients were encouraged to continue or begin participation in physical, occupational, and/or speech therapy (as appropriate based on the patient's diagnosis and baseline condition), which is thought to enhance and/or promote the effects of cell transplantation.

Efficacy Endpoint:

The change from baseline on neurologic and physical exam plus the following neurologic assessment tools conducted at 1, 3, 6, 9, 12 months post-treatment were available:
National Institutes of Health Stroke Scale (NIHSS) objectively quantifies the impairment caused by stroke in terms of level of consciousness, horizontal eye movement, visual field recognition, facial palsy, motor arm, motor leg, limb ataxia, sensory testing, language skill, speech.
Mini Mental Status Exam (MMSE) is a measure of cognitive impairment; an estimate of severity and progression of cognitive impairment, and the course of cognitive changes in an individual over time to document an individual's response to treatment.
Barthel Index (BI) of observed activity to establish degree of independence from any help, physical or verbal, however minor and for whatever reason.
The Geriatric Depression Scale (GDS) for discriminating between depressed and non-depressed subjects.

Study Enrollment

18 Subjects participated in this study for approximately 12 months as follows: up to 1 month for screening followed by treatment and follow-up.

Ischemic Tolerant Allogeneic Mesenchymal Bone Marrow Cells -itMSCs

Methods of preparing itMSCs for use in the present invention are disclosed in U.S. Pat. No. 8,790,081; Published U.S. Applications 2014/0286910; 2012/0201786, all of which are hereby incorporated by reference. In one aspect, the method of the invention employs itMSCs which are grown hypoxically in a defined serum medium, preferably a low serum medium of between about 0.2 and 2% serum.

Formulation, Packaging, And Preparation

On Day 1, the appropriate number of cryovials of allogeneic mesenchymal bone marrow cells per subject were thawed, washed and re-suspended in 10 ml of sterile 0.9% sodium chloride solution for injections according to the protocol. The final preparation for injection was made by diluting the allogeneic mesenchymal bone marrow cells into 100 ml 0.9% sodium chloride solution and used for intravenous dosing.

Dosage and Administration

Part 1 of this study was a dose escalation study evaluating three doses of stem cells, with subjects (n=5) receiving the same dose of allogeneic mesenchymal bone marrow cells based on weight per kilogram. Subjects were enrolled sequentially in a dose escalation manner, with subjects receiving one of three doses based on body weight and using the following cell aliquots: 25 million, 50 million, and 100 million allogeneic mesenchymal bone marrow cells, (Appendix A). No subjects received receive more than the maximum dosage of 150 million cells in this study.
Dose cohort 1: 0.5 million mesenchymal cells per kilogram body weight
Dose cohort 2: 1.0 million mesenchymal cells per kilogram body weight
Dose cohort 3: 1.5 million mesenchymal cells per kilogram body weight.

In Part 2, the dosage of stem cells is determined from the safety data from Part 1 and did not exceed the highest safe dose as determined in Part 1.

Administration of itMSCs

The itMSCs were prepared as described above. On Day 1, an intravenous line was placed into an appropriate vein on the upper extremity or hand with 0.9% sodium chloride solution running to keep the vein open. Prior to treatment, a skin test to bovine serum was administered using 0.1 ml aliquot of the Stem Cell Media (Bovine Growth Serum Hyclone.

Within 1 hour prior to infusion, the appropriate number of allogeneic mesenchymal bone marrow cells was diluted into 100 ml of 0.9% sodium chloride solution that was infused intravenously within 50 minutes.

Product Storage and Stability

The Master Cell Bank (MCB) and the lot release of allogeneic mesenchymal bone marrow cells were be stored in liquid nitrogen. The tolerable duration of storage was determined by qualified personnel as part of stability program. Once the vials of a lot release were processed and the cell suspension was prepared for administration to a subject as described above, the dose of allogeneic mesenchymal bone marrow cells was stored at +4° C. until the time of administration. All cells were administered to the subject within 2-4 hours of preparation. Cells that were not used within this time period were discarded.

Concomitant Medications and Rehabilitation

Concomitant medications (any prescription and/or over-the-counter preparations) and therapies (nondrug or procedures) used by a subject while participating in this clinical trial were recorded from 28 days prior to Dayl, until the end of the study or the end of a subject's participation in the study.

Physical, occupational, and speech therapy were allowed and encouraged for all subjects pre- and post-treatment. Specific therapeutic interventions were determined by the subject's treating physician based upon the patient-specific evaluation findings, impairments, treatment plans, and goals.

### STUDY ASSESSMENTS AND SCHEDULE

Clinical Assessments:
Complete medical history, including medication use
Standard physical examination, including vital signs
Neurologic examination

Clinical Laboratory Assessments:
Hematology: hemoglobin, hematocrit, platelet count, red blood cell count, white blood cell count, automated differential
Serum Chemistry: includes sodium, potassium, chloride, bicarbonate, blood urea nitrogen (BUN), creatinine, glucose, calcium, magnesium, phosphorous, uric acid, total and direct bilirubin, albumin and total protein, and the following liver function tests: (LFTs) alkaline phosphatase, alanine aminotransferase (ALT), aspartate aminotransferase (AST), immunoglobulin A (IgA), immunoglobulin E (IgE), immunoglobulin G (IgG), immunoglobulin M (IgM), and Lymphocyte Proliferation Panel
Coagulation: prothrombin time (PT), activated partial thromboplastin time (aPTT)
Urinalysis: urine dipstick for blood, protein, glucose, leukocyte esterase
Special Tests: hepatitis C antibody, hepatitis B surface antigen, HIV
Serum Pregnancy test: only for women of child-bearing potential

Imaging
Computerized Tomography (CT without contrast) of the brain.
CT with and without contrast of chest, abdomen and pelvis

Mental State Assessment Tools
National Institute of Health Stroke Scale (NIHSS)
Mini Mental Status Examination (MMSE)
The Barthel Index (BI)
Geriatric Depression Scale (GDS)

Screening

All screening evaluations were completed within 30 days prior to treatment, unless otherwise indicated.
Written informed consent
Inclusion/exclusion criteria review
Complete medical history, including medications
Standard physical examination, including vital signs (oral temperature, blood pressure, and pulse rate), weight, and height
Neurological examination
Neurologic assessment tools: NIHSS, BI, MMSE, GDS
12-lead ECG
HIV test
Hepatitis B surface antigen and hepatitis C antibody tests
Urine dipstick
   If positive the urine sample was sent for a complete urinalysis
Serum chemistry
LFT's, albumin and total protein
Prothrombin time (PT) and activated partial thromboplastin time (aPTT)
Complete blood count with differential (CBC)
Record concomitant medications
Brain C T (without contrast) is taken at the enrollment unless the CT or MRI was taken within 2 months of enrollment
CT with and without contrast of chest, abdomen and pelvis

Assessments and Treatment

Day-1 (Outpatient Admission)
Inclusion/exclusion criteria review
Reviewed and recorded any changes in medical and neurologic history, including concomitant medications from screening.
Confirmed Informed Consent Form (ICF) was complete with signature of patient, or legal guardian & witness.
Measured and recorded weight and vital signs (oral temperature, blood pressure, pulse rate) after subjects rest quietly in a supine or semi-recumbent position
Neurologic and physical examination
Target physical exam
Regular diet according to patient preference & hospital schedule
In the morning, the subject had an IV line placed in the upper extremity or hand with 0.9% sodium chloride running to keep the vein open, unless one was already in place
Bovine serum skin test prior to treatment (see Appendix B for description). If the participant tested positive on the Bovine serum skin test, he/she was excluded from the study.
Blood samples for IgA, IgE, IgG, IgM and for Lymphocyte Proliferation Panel (these samples were taken before patient treatment)
IV infusion of the prepared dose of allogeneic mesenchymal bone marrow cells diluted into 100 ml 0.9% sodium chloride within 50 minutes
Subjects were closely monitored for any signs of an infusion reaction
Measured and recorded vital signs every 2 hours, and as clinically indicated
Recorded adverse events and concomitant medications
Posted-treatment labs and results prior to discharge: Serum chemistry (specimen collected 1 hour after infusion complete) including BUN & Creatinine, CBC with diff, LFT's, PT, aPTT
12-lead ECG post treatment
Standard physical and neurologic exam prior to discharge
Recorded adverse events and concomitant medications
Patient was free to ambulate according to investigators discretion
The patient remained in the telemetry unit of the hospital for observation for 24 hours.
When clinically stable, patient was discharged home into the care of a responsible individual; however, if the patient was not considered clinically stable by the investigator, the patient was kept for observation until stable for discharge.
Provided patient or responsible individual accompanying the patient with the phone number for contacting the study nurse and/or investigator for any questions, concerns or changes in health status
Provide patient or responsible individual accompanying the patient a schedule for the follow-up assessments and tests.

Follow-up Assessments

Days 2, 3 and 4
Measured and recorded vital signs
Recorded adverse events and concomitant medication use
Targeted physical examination, focusing on the cardiac and respiratory systems
Neurologic examination (on day 4)
Obtained labs data: serum chemistry including BUN & Creatinine, CBC with diff, LFT's

Day 10 (±1 day)
Measured and recorded vital signs
Recorded adverse events and concomitant medication use
Standard physical examination
Neurologic examination
Serum chemistry including BUN & Creatinine, CBC with diff, LFT's

1 Month (Day 30±3 days)
Measured and recorded vital signs, and weight
12-lead ECG
Recorded adverse events and concomitant medication use
Standard physical examination
Neurologic examination
Serum chemistry including BUN & Creatinine, CBC with diff, LFT's
Neurologic assessment tools: NIHSS, BI, MMSE, GDS

Month 3 (Day 90±7 days)
Measured and recorded vital signs, and weight
Recorded adverse events and concomitant medication use
Neurologic examination
Standard physical examination
Neurologic assessment tools: NIHSS, the BI, MMSE, and GDS
Serum chemistry including BUN & Creatinine, CBC with diff, LFT's
Blood samples for IgA, IgE, IgG, IgM and for Lymphocyte Proliferation Panel

Month 6 (Day 180±10 days)
Measured and recorded vital signs, and weight
12-lead ECG
Recorded adverse events and concomitant medication use
Standard physical examination
Neurologic examination
Serum chemistry including BUN & Creatinine, CBC with diff, LFT's
Blood samples for IgA, IgE, IgG, IgM and for Lymphocyte Proliferation Panel
Neurologic assessment tools: NIHSS, BI, MMSE, GDS
CT with and without contrast of chest, abdomen and pelvis

Month 9 (Day 270±14 days)
Measured and recorded vital signs, and weight
Recorded adverse events and concomitant medication use
Neurologic examination
Standard physical examination
Neurologic assessment tools: NIHSS, BI, MMSE, GDS
Clinical labs: serum chemistry including BUN & Creatinine, CBC with diff, LFT's

Month 12 (Day 360±14 days)
Measured and recorded vital signs, and weight
Recorded adverse events and concomitant medication use
12-lead ECG
Urine dipstick
Neurologic examination
Standard physical examination
Neurologic assessment tools: NIHSS, BI, MMSE, GDS
Serum chemistry including BUN & Creatinine, CBC with diff, LFT's
Blood samples for IgA, IgE, IgG, IgM and for Lymphocyte Proliferation Panel
CT with and without contrast of chest, abdomen and pelvis

Early Termination Visit
Measured and recorded vital signs, and weight
Recorded adverse events and concomitant medication use
12-lead ECG
Standard physical examination
Neurologic examination
Serum chemistry including BUN & Creatinine, CBC with diff, LFT's, IgA, IgE, IgG, IgM
CT with and without contrast of chest, abdomen and pelvis
Optional pending subject agreement: neurologic assessment tools

Efficacy Analyses

The actual score and calculated change from baseline for each outcome measure (NIHSS, MMSE, BI, GDS) were tabulated for each scheduled assessment visit. Summary statistics 1 consisted of sample size (N), means, standard deviations, medians, and minimum and maximum values were used to summarize the data. Subjects who received complete dose of itMSCs and had post-treatment assessments were used as the efficacy evaluable population for statistical analysis.

The primary efficacy endpoint was evaluated as a change from baseline to month 6 on the neurologic assessment tools. Subjects missing the month 6 score, but having a score from month 9 or 12 were included in the analysis and used those available data.

The following are summaries of the results of the method of the present invention for treating mental state which involves administration intravenously of itMSCs to patients suffering from neurologic, functional, and/or motor deficits after ischemic stroke by intravenously administering ischemic tolerant allogeneic mesenchymal bone marrow cells (itMSCs).

NHISS Results

| | | |
|---|---|---|
| Total # Subjects with last postop | 12 | 67% |
| score improved (−) over baseline | | |
| p = | 0.666666667 | |
| SE = | 0.111111111 | |
| 95% CI = | 0.217777778 | |
| Upper CI | 88% | |
| Lower CI | 45% | |
| 1 point improvement | 1 | |
| 2 point improvement | 2 | |
| 3 point improvement | 4 | |
| 4 point improvement | 1 | |
| 5 point improvement | 2 | |
| 6 point improvement | 2 | |
| 7 point improvement | 0 | |
| 8 point improvement | 0 | |
| 9 point improvement | 0 | |
| 10 point improvement | 0 | |
| Total # Subjects with last postop | 3 | |
| score worse (+) than baseline | | |
| Total # Subjects with no change | 3 | |
| (0) from baseline | | |

Reference: NIH Stroke Scale Booklet National Institute of Neurological Disorders and Stroke.
Reference: Kwakkel, Gert, et al., J. Neurological Sciences, 294: Issues 1-2, pp. 57-61, Predictive Value of the NIHSS for ADL Outcome after Ischemic Hemisphericc Stroke: Does timing of early assessment matter? (2010)

MMSE Results

| | | |
|---|---|---|
| Total # Subjects with last postop | 13 | 76% |
| score improved (+) over baseline | | |
| p = | 0.764705882 | |
| SE = | 0.102879368 | |
| 95% CI = | 0.201643562 | |
| Upper CI | 97% | |
| Lower CI | 56% | |
| Total # 1 point + | 5 | |
| Total # 2 point + | 1 | |
| Total # 3 point + | 3 | |
| Total # 4 point + | 1 | |
| Total # 5 point + | 1 | |
| Total # 6 point + | 1 | |
| Total # 7 point + | 0 | |
| Total # 8 point + | 1 | |
| Total # 9 point + | 0 | |
| Total # 10 point + | 0 | |
| Total # Subjects with last postop | 0 | |
| score worse (−) than baseline | | |
| Total # Subjects with no change | 4 | |
| (0) from baseline | | |

Reference: “MINI-MENTAL STATE.” A Practical Method for Grading The Cognitive State of Patients for the Clinician. Journal of Psychiatric Research, 12(3): 189-198, 1975.

BI Results

| | | |
|---|---|---|
| Total # Subjects with last postop | 8 | 44% |
| score improved (+) over baseline | | |
| p = | 0.444444444 | |
| SE = | 0.117121395 | |
| 95% CI = | 0.229557934 | |
| Upper CI | 67% | |
| Lower CI | 21% | |
| 5 point improvement | 2 | |
| 10 point improvement | 3 | |
| 15 point improvement | 1 | |
| 20 point improvement | 1 | |
| >20 point improvement | 1 | |
| Total # Subjects with last postop | 5 | |
| score worse (−) than baseline | | |
| Total # Subjects with no change | 5 | |
| (0) from baseline | | |

GDS Results

| | | |
|---|---|---|
| Total # Subjects with last postop | 10 | 59% |
| score improved (−) over baseline | | |
| p = | 0.588235294 | |
| SE = | 0.119364625 | |
| 95% CI = | 0.233954665 | |
| Upper CI | 82% | |
| Lower CI | 35% | |
| 1 point improvement | 1 | |
| 2 point improvement | 1 | |
| 3 point improvement | 2 | |
| 4 point improvement | 1 | |
| 5 point improvement | 3 | |
| 6 point improvement | 1 | |
| 7 point improvement | 0 | |
| 8 point improvement | 0 | |
| 9 point improvement | 0 | |
| 10 point improvement | 0 | |
| >10 point improvement | 1 | |
| Total # Subjects with last postop | 5 | |
| score worse (+) than baseline | | |
| Total # Subjects with no change | 2 | |
| (0) from baseline | | |

Reference: Jerome A. Yesavage and Javaid Sheikh, Recent Evidence and Development of a Shorter Version; Clinical Gerontologist, Vol. 5, Issue 1-2, (1986); published online 25 Oct. 2008, pp. 165-173.

Table 1 is a summary of the efficacy resulting from use of the method of the present invention.

TABLE 1
Study in Ischemic Stroke Patients - Efficacy Results

| Efficacy Scale | Improvement | No Change | Worsening | 95% CI |
|---|---|---|---|---|
| NIHSS (N = 18) | 12/18 (67%) | 3/18 (17%) | 3/18 (17%) | 45%-88% |
| MMSE (N = 17) | 13/17 (76%) | 4/17 (24%) | 0/17 (0%) | 56%-97% |
| BI (N = 18) | 8/18 (44%) | 5/18 (28%) | 5/18 (28%) | 21%-67% |
| GDS (N = 17) | 10/17 (59%) | 2/17 (12%) | 5/17 (29%) | 35%-82% |

NIHSS—National Institute of Health Stroke
MMSE—Mini Mental State Examination
BI—Barthel Index
GDS—Geriatric Depression Scale
CI—confidence interval

The method of the present invention which involves a step of intravenously administering itMSCs to stroke patients achieved the following neurological benefits: in 10 of 17 patients, ameliorated depression as measured in changes in baseline of the Geriatric Depression Scale (Short Form); improved performance on the NIH Stroke Scale in 10 of 18 patients; improved performance on the MMSE in 13 of 17 patients; improved performance in 8 of 18 patients in the Barthel Index. In another aspect, the method of the invention finds application in treating the mental state of a post-ischemic stroke patient wherein the mental state comprises dementia.

### REFERENCES

1. Rosamond W, Flegal K, Furie K, Go A, Greenlund K, Haase N, Hailpern S M, Ho M, Howard V, Kissela B, Kittner S, Lloyd-Jones D, McDermott M, Meigs J, Moy C, Nichol G, O'Donnell C, Roger V, Sorlie P, Steinberger J, Thom T, Wilson M, Hong Y; for the American Heart Association Statistics Committee and Stroke Statistics Subcommittee. Heart Disease and Stroke Statistics 2008 Update. A Report from the American Heart Association Statistics Committee and Stroke Statistics Subcommittee. Circulation 2007 Dec. 17; [Epub].
2. Adams H, Adams R, Del Zoppo G, Goldstein L B; Stroke Council of the American Heart Association; American Stroke Association. Guidelines for the early management of patients with ischemic stroke: 2005 guidelines update: a scientific statement from the Stroke Council of the American Heart Association/American Stroke Association. Stroke 2005; 36:916-923.
3. Chamberlain G, Fox J, Ashton B, Middleton J. Concise Review: Mesenchymal stem cells: Their phenotype, differentiation capacity, immunological features, and potential for homing. Stem Cells, 2007; 25: 2739-2749.
4. Chen J, Zhang Z G, Li Y, Wang L, Xu Y X, Gautam S C, Lu M, Zhu Z, Chopp M. Intravenous administration of human bone marrow stromal cells induces angiogenesis in the ischemic boundary zone after stroke in rats. Circ Res 2003; 92:692-699.
5. Aleksandrova M A, Sukhih G T, Chailakhyan R K, Podgornyi O V, Marei M V, Poltavtseva R A, Gerasimov Y V. Comparative analysis of differentiation and behavior of human neural and mesenchymal stem cells in vitro and in vivo. Cell Technologies Biol Med 2006; 1:1-11.
6. Hermann A, Gastl R, Liebau S, Popa M O, Fiedler J, Boehm B O, Maisel M, Lerche H, Schwarz J, Brenner R, Storch A. Efficient generation of neural stem cell-like cells from adult human bone marrow stromal cells. J Cell Sci 2004; 117: 4411-4422.
7. Bai L, Caplan A, Lennon D, Miller R H. Human mesenchymal stem cells signals regulate neural stem cell fate. Neurochem Res. 2007; 32(2):353-62.
8. Aggarwal S, Pittenger M F. Human mesenchymal stem cells modulate allogeneic immune cell responses. Blood, 2005; 105:1815-22.
9. Nasef A, Mathieu N, Chapel A, Frick J, Francois S, Mazurier C, Boutarfa A, Bouchet S, Gorin N C, Thierry D, Fouillard L. Immunosuppressive effects of mesenchymal stem cells: involvement of HLA-G. Transplantation. 2007; 84(2):231-7.
10. Mylotte L A, Duffy A M, Murphy M, O'Brien R, Samali A, Barry F, Szegezdi E. Metabolic flexibility permits MSC survival in an ischemic environment. Stem Cells 2008 Feb. 28 [Epub].
11. Lazarus Hillard M; Koc Omer N; Devine Steven M; Curtin Peter; Maziarz Richard T; Holland H Kent; Shpall Elizabeth J; McCarthy Philip; Atkinson Kerry; Cooper Brenda W; Gerson Stanton L; Laughlin Mary J; Loberiza Fausto R; Moseley Annemarie B; Bacigalupo Andrea. Cotransplantation of HLA-identical sibling culture-expanded mesenchymal stem cells and hematopoietic stem cells in hematologic malignancy patients. Biology of blood and marrow transplantation: J of the American Society for Blood and Marrow Transplantation 2005; 11(5):389-98.
12. Koø O N, Day J, Nieder M, Gerson S L, Lazarus H M and Krivit W. Allogeneic mesenchymal stem cell infusion for treatment of metachromatic leukodystrophy (MLD) and Hurler syndrome (MPS-IR). Bone Marrow Transplantation (2002) 30, 215-222.
13. Horwitz E M, Gordon P L, Koo W K, Marx J C, Neel M D, McNall R Y, Muul L, and Hofmann T. Isolated allogeneic bone marrow-derived mesenchymal cells engraft and stimulate growth in children with osteogenesis imperfecta: Implications for cell therapy of bone. Proc Natl Acad Sci USA. 2002 Jun. 25; 99(13): 8932-8937
14. Le Blanc K, Rasmusson, I, Sandberg B, Gotherstrom C, Hassan M, Uzunel M, Ringden O. Treatment of severe acute graft-versus-host disease with third party haploidentical mesenchymal stem cells. Lancet (2004); 363(9419): 1439-41
15. Rabinovish S S, Seledtsov V I, Banul N V, Poveshchenko O V, Senyukov V V, Astrakov S V, Samarin D M, Taraban V. Ya. Cell Therapy of Brain Stroke. Cell Technologies in Biology and Medicine. 2005; 1.1: 25-28
16. Kondziolka D, Wechsler L, Goldstein S, Meltzer C, Thulborn K R, Gebel J, Jannetta P, DeCesare S, Elder E M, McGrogan M, Reiman M A, Bynum L. Transplantation of cultured human neuronal cells for patients with stoke. Neurology. 2000; 55:565-569
17. Gareth J, Lee S, Brosman C. Cytokines:powerful Regulators of Glial Cell Activation. Neuroscientist. 2003; 9-10
18. Wang L. Special issue: The intersection of stem/progenitor cell biology and hypoxic-ischemic cerebral injury/stroke. Experimental Neurology. Neuroscience Research 59. 2007; 327-340.
19. Horowitz E M, et al. Cytokines as the Major Mechanism of Mesenchymal Stem Cell Clinical Activity: Expanding the Spectrum of Cell Therapy. Israel Medical Association World Fellowship Conference. 2009; 11:209-211.
20. Deans R J, Moseley A B. Mesenchymal Stem Cells; Biology and Potential Clinical Uses. Experimental Hematology. 2000; 28:875-884.
21. Honczarenko, M et al. Human Bone Marrow Stromal Cells Express a Distinct Set of Biologically Functional Chemokine Receptors. Stem Cells. 2006; 24:1030-1041.
22. Crigler L, et al. Human Mesenchymal Stem Cell Subpopulations Express a Variety of Neuro-Regulatory Molecules and Promote Neuronal Cell Survival and Neuritogenesis. Experimental Neurology. 2006; 198:54-64.
23. Sondell M, et al. Vascular Endothelial Growth Factor is a Neurotrophic Factoe Which Stimulates Axonal Outgrowth Through flk-1 Receptor. European Journal of Neuroscience. 2000. December; 12(12):4243-54.
24. Silverman W F, et al. Vascular, Glial and Neuronal Effects of Vascular Endothelial Growth Factor in Nesencephalic Explant Cultures. Neuroscience. 1999; 90(4):1529-1541.
25. Jin K, et al. Heparin-Binding Epithelial Growth Factor-Like Growth Factor: Hypoxia-Inducible Expression in Vitro and Stimulation of Neurogenesis in Vitro and in Vivo. The Journal of Neuroscience. 2002; 22(13):5365-5373.
26. Jin k, et al. Vascular Endothelial Growth Factor (VEGF) Stimulates Neurogenesis in Vitro and in Vivo. Proceedings of the National Academy of Sciences. 2002. 99(18):11946-11950.
27. Matsuzaki H, et al. Vascular Endothelial Growth Factor Reserves Hippocampal Neurons from Glutamate-Induced Toxicity: Signal Transduction Cascades. The Journal of the Federation of American Societies for Experimental Biology. 2001. May; 15(7):1218-1220.
28. Ogunshola O O, et al. Panacrine and Autocrine Functions of Neuronal Vascular Endothelial Growth Factor (VEGF) in the Central Nervous System. Journal of Biological Chemistry. 2002. Mar. 29; 277(13):1140-5.
29. Oosthyse B, et al. Deletion of the Hypoxia-Response Element in the Vascular Endothelial Growth Factor Promoter Causes Motor Neuron Degeneration. Nature Genetics. 2001. June; 28(2):131-8.

### REFERENCES

Mahoney F I. Barthel D. “Functional evaluation: the Barthel Index” Maryland State Medical Journal 1965; 14:56-61. Used with permission.
Loewen S C, Anderson B A. “Predictors of stroke outcome using objective measurement scales.” Stroke, 1990; 21:78-81.
Gresham G E, Phillips T F. Labi M L. “ADL status in stroke: relative merits of three standard indexes.” Arch Phys Med Rehabit. 1980; 61:355-358.
Collin C. Wade D T. Davies S, Home B. “The Barhel ADI. Index: a reliability study.” Int Disability Study, 1988; 10:61-63.

1. A method of treating the mental state of an injured patient comprising the step Ls of:
(a) intravenously administering to a patient in need an effective amount of allogenic, bone marrow-derived ischemic tolerant mesenchymal stem cells (itMSCs) to said patient; and
(b) measuring said patient's performance score(s) on mental state assessment tools relative to said patient's performance score(s) on said assessment tools prior to step (a).

2. The method of claim 1 wherein said mental state comprises one or more of motor, functional, and neurologic impairment.

3. The method of claim 1 wherein said patient in need has ischemic brain injury.

4. The method of claim 3 wherein said injury is stroke.

5. The method of claim 1 wherein said mental state comprises depression.

6. The method of claim 1 wherein said mental state comprises dementia.

7. A method of improving an injured patient's performance relative to a baseline score on one or more mental state assessment tests selected from the group consisting of the National Institutes of Health Stroke Scale (NIHSS), Mini Mental Status Exam (MMSE), Barthel Index (BI), and The Geriatric Depression Scale (GDS),said method, comprising the steps of:
(a) intravenously administering to a patient in need an effective amount of allogenic, bone marrow-derived ischemic tolerant mesenchymal stem cells (itMSCs), and
(b) measuring said patient's performance score(s) on said one or more mental state assessment tools relative to said patient's performance score(s) on said assessment tools prior to step (a).

8. The method of claim 7 wherein said patient in need has a phenotype comprising one or more of motor, functional and neurological impairment.

9. The method of claim 7 wherein said patient has ischemic brain injury.

10. The method of claim 7 wherein said injury is stroke.

11. The method of claim 7 wherein said improving relative to a baseline score in performance comprises amelioration of depression.

12. The method of claim 7 wherein said improving relative to a baseline score in performance comprises amelioration of dementia.

13. A method of inducing neurological benefits in patients with neurologic impairment comprising the steps of:
(a) intravenously administering to a patient in need an effective amount of allogenic, bone marrow-derived ischemic tolerant mesenchymal stem cells (itMSCs); and
(b) measuring said patient's performance score(s) on mental state assessment tools relative to said patient's performance score(s) on said assessment tools prior to step (a).

14. The method of claim 13 wherein said benefit is amelioration of depression.

15. The method of claim 13 wherein said benefit is decrease of impairment in one or more of the domains consisting of level of consciousness, horizontal eye movement,
visual field recognition, facial palsy, motor arm, motor leg, limb ataxia, sensory testing, language skill, and speech.

16. The method of claim 13 wherein said benefit is reduction of cognitive impairment.

17. The method of claim 13 wherein said benefit is improved activity to establish degree of independence from any help, physical or verbal, however minor and for whatever reason.

18. The method of claim 13 wherein said benefit is amelioration of dementia.
